(19) United States
(12) Patent Application Publication
(10) Pub. No.: US 2003/0177590 A1
(43) Pub. Date: Sep. 25, 2003

(54) Aqueous dispersion and method of use

(75) Inventors: Isabelle Rollat-Corvol, Paris (FR); Aude Livoreil, Paris (FR); Gabin Vic, Compiegne (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/369,767

(22) Filed: Feb. 21, 2003

Related U.S. Application Data
(60) Provisional application No. 60/362,088, filed on Mar. 7, 2002.

(30) Foreign Application Priority Data
Feb. 21, 2002 (FR) 0202187

(51) Int. Cl. A61K 7/13

(52) U.S. Cl. 8/405; 8/406; 8/407; 8/426

(57) ABSTRACT

This invention relates generally to the use, for example in the cosmetics field and in particular for the treatment and styling of the hair, of an aqueous dispersion of particles of the nucleus/casing (core/shell) type containing compounds with reactive silyl functions, as well as to a process for treatment and a process for styling of the hair using such an aqueous dispersion. The dispersions and particles also make up a part of this invention, as do their methods of manufacture.

### REFERENCE TO PRIOR APPLICATIONS

This application claims priority to U.S. provisional application No. 60/362,088 filed Mar. 7, 2002 and to French patent application 0202187 filed Feb. 21, 2002, both incorporated herein by reference in their entirety.

### SUMMARY OF THE INVENTION

This invention relates generally to the use, for example in the cosmetics field and in particular for the treatment and styling of the hair, of an aqueous dispersion of particles of the nucleus/casing (core/shell) type containing compounds with reactive silyl functions, as well as to a process for treatment and a process for styling of the hair using such an aqueous dispersion. The dispersions and particles also make up a part of this invention, as do their methods of manufacture.

### BACKGROUND OF THE INVENTION

There are numerous hairstyling products which make it possible to arrange the hair and to hold it in place for a certain time. Nonetheless, the hairstyling effect of these products generally lasts scarcely longer than one day, because the products are removed by washing or by brushing of the hair at the end of the day.

The formation on fibrous substrates and in particular on the hair, of enduring silicone films resistant to several shampooings recently has been proposed in the patent application WO 98/54255 (and in the corresponding U.S. Pat. No. 6,326,011). The endurance of the deposits is due to an in situ crosslinking of polymers bearing reactive alcoxysilane functions in an aqueous medium. The fact that the crosslinking of the polymers is induced by the presence of water, however, requires the storage of the crosslinkable compositions in an anhydrous environment, protected from atmospheric humidity, in order to prevent a premature crosslinking prior to deposition. In addition, the process for treatment of the hair with these compositions is relatively complex because it requires a stage of application of water prior to or following the stage of application of the crosslinkable anhydrous product.

### DETAILED DESCRIPTION OF THE INVENTION

The inventors have discovered that it is possible to overcome at the same time the drawbacks resulting from the use of non-enduring hairstyling products and those resulting from the use of enduring hairstyling products crosslinkable by hydrolysis, disclosed in WO 98/54255, by using for the treatment and styling of the hair aqueous dispersions of particles having a nucleus/casing (core/shell) structure and containing in their nucleus compounds with reactive silyl functions. These aqueous dispersions are stable over the course of time, that is to say they do not deposit sediment or crosslink during storage, but are capable, after deposition and drying, of crosslinking in situ and therefore of forming enduring crosslinked coatings, that is to say coatings which are not removed with brushing or shampooing.

This invention thus provides a new use of an aqueous dispersion of particles of the nucleus/casing type having a casing (shell, envelope, etc.) comprising at least one acrylic polymer, and a core, or nucleus, etc., comprising at least one compound with a reactive silyl function. The invention use is preferably one in the cosmetics industry, and is more preferably one for the treatment and styling of human keratinous fibers. The dispersion and particles themselves, and their methods of manufacture, also make up a part of the invention.

One embodiment of the invention process is the treatment of human keratinous fibers, in particular hair, comprising

the application of such an aqueous dispersion on dry or dampened hair, and

the drying of the hair in the open air at room temperature or by means of a drying device such as a hairdryer,

as well as a process for styling of the hair further comprising, following application of the dispersion and prior to drying, the arranging of the treated hair.

In another embodiment the invention provides a process for pretreatment or post-treatment of human keratinous fibers comprising effecting the above treatment process before or after a coloring or color-stripping or a perming or straightening treatment.

The inventors have noted with surprise that the coatings formed in this manner had a granular structure on a micrometric scale which was expressed by a surface roughness and a non-slip effect. At the time of application testing, it turned out that the non-slip effect facilitated the arranging or rearranging of the hair, and particularly of fine hair. The hair has more body and is easier to manage. The roughness of the deposits formed, however, does not impair the cosmetic qualities of the treated hair, which does not have a dull appearance and retains a relatively smooth feel.

As indicated above, the deposits formed are enduring and the hairstyling effect is retained for at least one shampooing. In addition, the process for treatment or hairstyling using these dispersions is simpler than the one disclosed in WO 98/54255 (and U.S. Pat. No. 6,326,011), because it does not necessarily involve moistening of the hair prior to application of the treatment or hairstyling composition or the addition of water in the anhydrous composition.

The compounds with reactive silyl functions, present in the nucleus of particles of the nucleus/casing type, preferably are chosen from among those of formula (I)

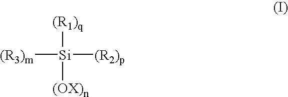

in which

R₁ represents a polymeric residue or an R₃ group,

R₂ represents a C₁₋₈ alcoxy group,

R₃ represents a linear, branched or cyclic C₁₋₈ alkyl group,

X represents a hydrogen atom or a cation of a base used for the neutralization of silanol groups,

m represents a whole number ranging from 0 to 3,

q has the value of 0 or 1,

n and p each represent a whole number ranging from 0 to 4,

the sum of n+p has the value of 1, 2, 3 or 4, and

the sum of m+n+p+q has the value of 4.

R₂ preferably represents a methoxy or ethoxy group and R₃ represents a methyl or ethyl group. When R₁ represents a polymeric residue, the latter preferably is of the acrylic type bearing anionic charges.

For the neutralization of the silanol functions, any (preferably cosmetically) acceptable organic or inorganic base whatsoever may be used, and ammonia and the alkaline metal hydroxides, in particular sodium or potassium hydroxide, are particularly preferred. When a cation is involved, X therefore preferably represents an alkaline metal atom or the NH₄⁺ ion.

The acrylic polymers of the casing preferably are anionic acrylic polymers, that is to say acrylic polymers comprising moieties derived from acrylic acid and/or methacrylic acid neutralized with a base. They are preferably essentially insoluble in water.

While not bound by theory, the rough structure of the deposits which imparts a hairstyling capacity thereto is attributed to the existence, in the aqueous dispersions used in this invention, to totally distinct particles, separated from each other, which at least partially retain their form in the deposit obtained.

To achieve deposits with a sufficient hairstyling effect, but which afford satisfactory cosmetic properties (smooth feel, sheen), the mean size of the organic copolymer particles in aqueous dispersion (measured by light diffusion) preferably ranges between 10 nm and 1000 nm and in particular between 50 nm and 500 nm, these broad ranges including all values and subranges therebetween and endpoints thereof, as if specifically written out. For example, included are 15, 20.5, 345, 678.9, 566-789, 850 nm, etc.

An aqueous dispersion of particles is available on the market under the name SANMOL® EW 102 (Sanyo Chemical), the technical brochures thereof being incorporated herein by reference.

The aqueous dispersions used in accordance with this invention, useful in the cosmetics field and in particular in the field of styling and care of the hair, preferably have a content of particles of the nucleus/casing type containing compounds with silyl functions ranging between 1 and 80% by weight, in particular between 1 and 50% by weight, in relation to the total weight of the dispersion, all values and subranges therebetween being specifically included.

Another embodiment of his invention is a composition comprising, preferably in a cosmetically acceptable medium, from 1 to 99% by weight, more preferably from 1 to 50% by weight, all values and subranges between these ranges being included, of a stable aqueous dispersion of particles of the nucleus/casing type containing compounds with reactive silyl functions and one or more hydrosoluble polymers, synthetic or of natural origin.

For reasons of stability of the compositions, their pH preferably is in excess of or equal to 5, in particular ranging between 5 and 11, and more preferentially between 7 and 11.

Useful hydrosoluble polymers, synthetic or of natural origin, may be chosen for example, from among polyvinylpyrrolidone, poly(acrylic acid), poly(vinyl alcohol), polyacrylamide, poly(acrylamidomethylpropane sulfonic acid) or copolymers thereof, crosslinked or non-crosslinked, non-ionic guar gums, biopolysaccharide gums of microbial origin such as scleroglucane and xanthane gums, gums deriving from vegetable exudates such as gum arabic, ghatti gum, karaya gum or tragacanth gum, gums extracted from seaweed such as carrageens or agar, gums deriving from vegetable extracts such as carob gum or the pectins extracted from fruit pulps, alginates, starches and hydroxyalkylcelluloses and carboxyalkylcelluloses. These polymers may be anionic, cationic, non-ionic or amphoteric. They are preferably present in the compositions of this invention in the amount of 0.01 to 30% by weight.

The compositions of this invention also may contain one or more cosmetic additives or additives with a formulation customarily used in the cosmetic field, and in particular in the field of styling and care of the hair. As examples of such additives there may be cited UV filters, fragrances, preservatives, pigments and coloring agents, anti-foaming agents, vitamins, conditioners such as soluble silicones, dispersed or microdispersed, agents for adjustment and stabilization of pH, synthetic or non-synthetic organic or inorganic particles, anionic, cationic, non-ionic or amphoteric surfactants.

The individual skilled in the art of course will choose these additives making sure that their presence in the compositions of this invention does not significantly impair the intrinsic advantageous properties of these compositions, in particular the dispersed state of the particles, the stability of the compositions in storage and the reactivity of the silyl functions at the time of drying of the cosmetic deposits.

The compositions according to this invention may take any form whatsoever compatible with the existence of an aqueous dispersion of particles of an organic polymer. As an example there may be cited lotions packaged in a pump bottle or in an aerosol can, or thickened or even gelled suspensions.

The following examples illustrate this invention, without being limiting.

### EXAMPLE 1

Preparation of a Hairstyling Composition and Stability in Storage

A hairstyling composition A is prepared by mixing the following ingredients:

| | |
|---|---|
| SANMOL ® EW102^{a)} | 10% |
| water | 100% |
| base (NH₄OH) | q.s.p. pH 8.5 |

^{a)}aqueous dispersion according to the invention marketed by the company Sanyo Chemical.

In order to evaluate the stability of this composition in storage, the mean size of the particles is measured with the aid of a light diffusion apparatus (Coulter model N4S, marketed by the company Coultronics)

| | |
|---|---|
| • at the time of preparation of composition A: | 156 nm |
| • after 1 month of storing at room temperature: | 158 nm |
| • after 2 months of storing at a temperature of 45° C.: | 149 nm |

The results obtained show that there occurs no particle agglomeration or condensation phenomenon which would be expressed by an increase in the mean particle size measured. The composition therefore is completely stable.

### EXAMPLE 2

Formation of a Deposit by Drying in the Open Air

25 mg/cm² of the SANMOL® EW 102 dispersion is deposited in a Petri dish and allowed to dry in the open air. The surface of the dried film is analyzed by atomic force microscopy (AFM), in friction mode, with an applied friction force of 10 nN. The cantilever performs 256 scannings of the surface at the rate of 1 scanning per second.

It is noted that the deposit has a rough structure on which small bumps uniformly distributed over the entire surface are perceived. The film is slightly opalescent.

### EXAMPLE 3

Evaluation of the Hairstyling Effect and the Cosmetic Properties

Composition A of example 1 is applied, by a group of 20 individuals, by means of a pump bottle, to still-damp washed hair. After it has been on for approximately five minutes, the individuals style their hair in their usual manner.

The table below indicates the number of individuals, among the 20 interviewed, evincing an impression of improvement in ease of hairstyling and in cosmetic properties such as volume or texture of the hairstyle, on the day of application, after 1 shampooing.

| | management | volume | texture | holding |
|---|---|---|---|---|
| | of | of | of | of |
| | hairstyle | hairstyle | hairstyle | hairstyle |
| day of | 14 | 15 | 14 | 15 |
| application | | | | |
| after 1 | 17 | 13 | 13 | 16 |
| shampooing | | | | |

Composition A according to the invention therefore makes possible an easier hairstyling with holding of the hair and an improvement in management and volume which are clearly perceived by the individuals having participated in the test.

### EXAMPLE 4

Hairstyling Composition

There is prepared a hairstyling composition B, identical to the hairstyling composition A of example 1, apart from the fact that it also contains 0.08% by weight of a hydrosoluble polymer, that is, a hydroxypropyl guar gum, marketed under the name Jaguar® HP 105 by the company Rhodia Chimie.

In a test similar to the one described in example 3, conducted with a group of 10 individuals, this composition produces effects equivalent to those obtained with composition A.

The hairstyling effect and the cosmetic properties are even slightly better. In particular, the hair is smoother and softer.

### EXAMPLE 5

Hairstyling Composition

There is prepared a hairstyling composition identical to that of composition A according to example 1, apart from the fact that it also contains 0.4% by weight of poly(vinyl alcohol) (Airvol® 540, marketed by the company Air Products Chemical Inc.).

In a test similar to the one described in example 3, conducted with a group of 10 individuals, this composition produces effects equivalent to those obtained with composition A. The hairstyling effect and the cosmetic properties are even slightly better. In particular, the hair is smoother and softer. This latter effect is especially apparent for damaged hair.

### EXAMPLE 6

Effect of the pH of the Composition

In a test similar to the one described in example 3, composition A (pH 8.5) is compared with a composition D identical apart from the fact that it has a pH of 10.5 (adjusted by addition of NH₄OH).

It is noted that the hairstyling effect and the volume of the hairstyle provided by composition D are superior to those obtained with composition A, in particular on damaged hair, but that composition A provides more management and a more pleasing feel.

This example shows that it is possible to adjust the properties of the hairstyling compositions of this invention by means of a mere modification of the pH.

The above provides a written description of the invention, and of the manner and method of making and using it, such that it enables any person skilled in the art to make and use the same, this enablement being provided in addition for a method for treating human keratinous fibers, comprising applying thereto an aqueous dispersion of particles having a nucleus/casing structure, wherein said casing comprises an acrylic polymer, and wherein the nucleus comprises a compound having a reactive silyl function, these compounds having a reactive silyl function preferably being selected from the group of compounds having formula (I) described above. Also enabled is an aqueous dispersion of particles having a nucleus/casing structure, wherein the casing comprises an acrylic polymer insoluble in water, and the nucleus comprises a compound having a reactive silyl function and one or more hydrosoluble polymers, as well as a process for the cosmetic treatment of human keratinous fibers, comprising

applying an aqueous dispersion of particles having a nucleus/casing structure, wherein said casing comprises an acrylic polymer, and wherein the nucleus comprises a compound having a reactive silyl function to dry or dampened fibers to produce coated fibers, and

drying the coated fibers at room temperature or by applying heat, and a process for the cosmetic treatment of human keratinous fibers,

and a process comprising

applying an aqueous dispersion of particles having a nucleus/casing structure, wherein the casing comprises an acrylic polymer insoluble in water, and the nucleus comprises a compound having a reactive silyl function and one or more hydrosoluble polymers to dry or dampened fibers to produce coated fibers, and

drying the coated fibers at room temperature or by applying heat.

All documents, references, texts, patents, applications, publications, tests, brochures, etc mentioned herein are expressly incorporated herein by reference. Stated ranges always include the endpoints thereof, and include all subranges and values therebetween.

1. A method for treating human keratinous fibers, comprising applying thereto an aqueous dispersion of particles having a nucleus/casing structure, wherein said casing comprises an acrylic polymer, and wherein the nucleus comprises a compound having a reactive silyl function.

2. The method according to claim 1, wherein said fibers are hair.

3. The method according to claim 1, wherein the compound having a reactive silyl function is selected from the group of compounds having formula (I): 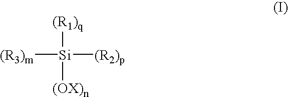
in which
R₁ represents a polymeric residue or an R₃ group,
R₂ represents a C₁₋₈ alcoxy group,
R₃ represents a linear, branched or cyclic C₁₋₈ alkyl group,
X represents a hydrogen atom or a cation of a base used for the neutralization of silanol groups,
m represents a whole number ranging from 0 to 3,
q has the value of 0 or 1,
n and p each represent a whole number ranging from 0 to 4,
the sum of n+p has the value of 1, 2, 3 or 4, and
the sum of m+n+p+q has the value of 4.

4. The method according to claim 3, wherein R₃ represents a methyl or ethyl group.

5. The method according to claim 3, wherein R₂ represents a methoxy or ethoxy group.

6. The method according to claim 1, wherein said acrylic polymer is anionic, essentially insoluble in water, and comprises polymerized moieties of acrylic acid and/or methacrylic acid neutralized by a base.

7. The method according to claim 3, wherein R₁ denotes an anionic acrylic polymeric residue.

8. The method according to claim 1, wherein a mean size of the particles is from 10 nm to 1000 nm.

9. The method according to claim 1, wherein the concentration of particles in the dispersion is from 1% to 80% by weight, in relation to the total weight of the dispersion.

10. An aqueous dispersion of particles having a nucleus/casing structure, wherein the casing comprises an acrylic polymer insoluble in water, and the nucleus comprises a compound having a reactive silyl function and one or more hydrosoluble polymers.

11. The dispersion according to claim 10, wherein the concentration of hydrosoluble polymer or polymers ranges between 0.01 and 30% by weight in relation to the total weight of the composition.

12. The dispersion according to claim 10, having a pH in excess of or equal to 5.

13. The dispersion according to claim 10, further comprising a cosmetic hair styling and care additive.

14. A process for the cosmetic treatment of human keratinous fibers, comprising
applying an aqueous dispersion of particles having a nucleus/casing structure, wherein said casing comprises an acrylic polymer, and wherein the nucleus comprises a compound having a reactive silyl function to dry or dampened fibers to produce coated fibers, and
drying the coated fibers at room temperature or by applying heat.

15. The process of claim 14, wherein said aqueous dispersion is applied before or after a coloring, color stripping, perming or straightening treatment.

16. A process according to claim 14, comprising:
applying an aqueous dispersion of particles having a nucleus/casing structure, wherein said casing comprises an acrylic polymer, and wherein the nucleus comprises a compound having a reactive silyl function to dry or dampened hair to produce coated fibers,
arranging the coated fibers, and
drying the coated fibers at room temperature or by applying heat.

17. A process for the cosmetic treatment of human keratinous fibers, comprising
applying an aqueous dispersion of particles having a nucleus/casing structure, wherein the casing comprises an acrylic polymer insoluble in water, and the nucleus comprises a compound having a reactive silyl function and one or more hydrosoluble polymers to dry or dampened fibers to produce coated fibers, and
drying the coated fibers at room temperature or by applying heat.

18. The process of claim 17, wherein said aqueous dispersion is applied before or after a coloring, color stripping, perming or straightening treatment.

19. A process according to claim 17, comprising:
applying an aqueous dispersion of particles having a nucleus/casing structure, wherein the casing comprises an acrylic polymer insoluble in water, and the nucleus comprises a compound having a reactive silyl function and one or more hydrosoluble polymers to dry or dampened hair to produce coated fibers,
arranging the coated fibers, and
drying the coated fibers at room temperature or by applying heat.
